(19) United States
(12) Patent Application Publication
(10) Pub. No.: US 2009/0012569 A1
(43) Pub. Date: Jan. 8, 2009

(54) Configurable Bone Fixation System

(76) Inventors: Desmond Meiring Dall, Hermanus (ZA); Anthony William Miles, Bath (GB)

Correspondence Address: STITES & HARBISON PLLC, 1199 NORTH FAIRFAX STREET, SUITE 900, ALEXANDRIA, VA, 22314, US

(21) Appl. No.: 11/908,875

(22) PCT Filed: Mar. 16, 2006
(86) PCT No.: PCT/GB2006/000940

§ 371 Date: May 1, 2008

(30) Foreign Application Priority Data
Mar. 17, 2005 (GB) 0505505.8

(51) Int. Cl. A61B 17/70; A61B 17/56

(52) U.S. Cl. 606/280; 606/297; 606/60

(57) ABSTRACT

A bone fixation system has an elongate portion (122; 299) for engaging over a long bone (e.g. a femur) and an end engagement portion (120; 260) for engaging over an end region of a long bone (e.g. a greater trochanter). To allow for variation in bone geometries, the two portions are pivotably connectable. Preferably the connection is lockable. Thus the system can be adapted to a particular bone geometry and then made rigid.

### TECHNICAL FIELD

The present invention relates to a configurable bone fixation system, namely to surgical apparatus and methods for use in the treatment of bones and joints. It is particularly, though not exclusively, concerned with systems for use in hip replacement operations such as attachment of trochanter and/or proximal femur reconstruction, revision hip surgery or when an extended or Wagner osteotomy approach is used. It is also relevant to other anatomical regions, particularly regions involving a long bone and an end region thereof. For example, it has application to the proximal tibia, distal femur, and the proximal and distal humerus regions.

### BACKGROUND ART

The present inventors have previously devised many components of use to surgeons. For example, U.S. Pat. Nos. 4,269,180 and 6,066,141 describe bone grips. These are curved hook-like elements, primarily for engaging over a part of a greater trochanter which has been cut (osteotomised) and temporarily removed during a hip replacement operation, and which is being replaced. U.S. Pat. No. 5,665,089 discloses a range of plate-like elements for attachment to a femur for use in bone implant surgery such as hip replacements, or in subsequent surgical treatment of the bone structure in the region of an implant.

More recently, use has been made of components which combine a bone grip (particularly for engaging over a trochanter), and a plate portion, for extending down over the femur. For example, Zimmer produced the “Cable-Ready” (registered trademark) greater trochanteric reattachment system. This involves a component which has a straight, flat, elongate plate portion, integral with a hooked portion, terminating in a spike. Ideally, the hooked grip portion lies over the greater trochanter, and the plate portion overlies the shaft of the femur. Both portions have apertures to receive cerclage cables, which are passed around the bone, to secure the device in place.

However, there is a considerable variation in size and shape of trochanters, and of the proximal femoral shaft regions, and in the angular relationships between them. FIGS. 1A to 1D show the upper end of a femur 10 which has received a new hip joint ball assembly 12. Near the top of the femur, there is a large bulge projecting upwardly and outwardly. This is the greater trochanter 14. As mentioned above, it is commonly cut during hip replacement surgery, and subsequently reattached by means of some form of bone grip. FIGS. 1A and 1B show examples of the variation in the size and shape of the greater trochanter. (There is also a smaller bulge pointing medially inwardly; this is the lesser trochanter 16.) FIGS. 1A to 1D show a grip plate 18, having an upper grip portion 20 which serves as a bone grip, engaging over the greater trochanter 14; and an elongate plate portion 22 which is intended to extend along the femur and be attached to it. As can be seen from the anterior views of FIGS. 1C and 1D, it can be difficult to fit a standard grip plate 18 to a range of different femurs which may have different forms of asymmetry (FIG. 1C having a left anterior bow and FIG. 1D showing a right anterior bow).

It is known to provide a range of different components, e.g. differing in whether the plate portion is in line with the grip portion or connected with a “right-hand” or “left-hand” twist. There may also be variations in the size and shape of the hook portions etc. However, not only does this add considerable complication to the manufacture and supply and use of such components, it can at best be only a rather partial solution.

### DISCLOSURE OF INVENTION

Broadly, in a first aspect, the present invention provides a surgical component having an elongate first portion for engaging over an elongate bone (which may be, for example, a femur), and a second, end engagement portion for engaging an element associated with an end region of the bone (which may be, for example, the greater trochanter), wherein the first component is hingedly connected to the second component.

Generally at least one, and usually both, of the first and second portions are adapted to be connected to bone, e.g. having apertures for bone screws and/or being adapted for use with a cerclage system. Thus these may be through-bores or other guide formations for cerclage cables. These may be bore portions that are crimpable to lock a cerclage cable portion therein. There may be alternative fastening systems usable with polymer cerclage cables, e.g. buckle systems.

The term “hingedly connected” means that the orientation of one component relative to the other can be varied. This may be because of a connecting region which permits bending, or because of a form of mechanical connection that permits relative movement, for example a pivot or a hinge pin. There may be an intermediate portion which is hinged at respective spaced locations to the first and second portions, allowing a greater degree of hingeing in one or more directions. A hingeing device may be lockable. (It is not generally desirable for the components to undergo relative movement after the surgical procedure has been completed.) Hinged portions may be separable. Thus, the invention envisages a modular system, in which a surgeon may choose from a range of first portions and a range of second portions which can be connected together hingeably.

In another aspect the invention provides an end engagement element, generally a bone grip, having a bone engagement portion and a projecting connection portion. The element is engageable with an end portion of a bone (e.g. a greater trochanter). The projecting connection portion may then extend in the main direction of extension of the bone. The projecting connection portion may have an aperture, preferably threaded. This can receive a fixing element, e.g. a bone screw, for fixing it to bone. Alternatively it can receive a fixing element, e.g. a locking screw, for connecting it to an elongate first portion of a surgical component, as referred to above. Thus it can be part of a modular system. The projecting connection portion may have one or more transverse bores providing paths for cerclage cables, or other adaptations to cerclage systems as referred to above. It may be “hingedly connected” to the bore engagement portion, e.g. via a thinned neck portion.

The mode of connection may also allow some length adjustment.

The invention will now be described in more detail with reference to the accompanying drawings. These show devices intended for engaging the greater trochanter and femur, generally in the context of a hip replacement operation or revision procedures. However, the invention is not limited to such procedures or to such sites.

### BRIEF DESCRIPTION OF DRAWINGS

FIGS. 1A, 1B, 1C and 1D are schematic partial views of femurs, including the greater trochanter, with attached grip plates, showing variations in size and shape of the femur.

FIGS. 2A and 2B are schematic perspective and front elevation views of an extended trochanter grip plate embodying the invention.

FIGS. 2C and 2D show variant plate portions, and

FIG. 2E shows the grip plate in position on a trochanter and a femur.

FIGS. 3A and 3B are partial front and side elevations of a grip plate embodying the invention and which employs a flexible junction.

FIGS. 4A and 4B are a pair of views similar to 3A and 3B but showing an assembly that hinges by means of a hinge pin.

FIGS. 5A and 5B are a similar pair of drawings showing a grip plate which hinges via an intermediate portion with two hinge pins.

FIG. 6A shows a design similar to that shown in FIG. 4A using a single hinge pin.

FIG. 6B shows a detail of a variant incorporating a lock nut.

FIGS. 7 and 8 show variations of the design of FIG. 6A.

FIG. 9 is a view similar to FIG. 6A showing a variant using a shaped hinge pin to allow a further degree of rotational freedom.

FIGS. 10A and 10B show a design in which the components are connectable via a universal joint.

FIGS. 11A and 11B are views showing a double waisted hinge region.

FIG. 12A shows a design with an intermediate portion in the form of a circular link. FIG. 12B shows an alternative oval link.

FIGS. 13A and 13B show a design similar to FIGS. 5A and 5B, employing an intermediate piece and two hinge pins.

FIGS. 13C, 13D and 13E show variant link pins.

FIGS. 14A and 14B are a pair of views of a device using a slotted pivot connection, FIG. 15B being partly in section.

FIG. 15 is a plan view showing a detail of FIG. 14B as viewed in the direction of the arrow A.

FIGS. 16A and 16B show a pivoting connection using a hook and eye.

FIG. 17A is a front view showing a form of connection using a hook engaging a pivot shaft, and FIG. 17B is a section on XVII-XVII.

FIGS. 18A and 18B are views showing a locking hinge design, FIG. 18C is an exploded view of the locking hinge components, and FIG. 18D is a view of a component in direction X marked in FIG. 18C.

FIGS. 19A and 19B are views showing a bi-directional locking hinge design.

FIGS. 20A and 20B show an end engagement element with a projecting connection portion. FIG. 20C is an enlarged sectional view on Z-Z in FIG. 20A, also showing a bone screw.

FIG. 21A shows the end engagement element of FIG. 20A connected to a bone plate by a locking screw, and FIG. 21B is a sectional view of the connection region.

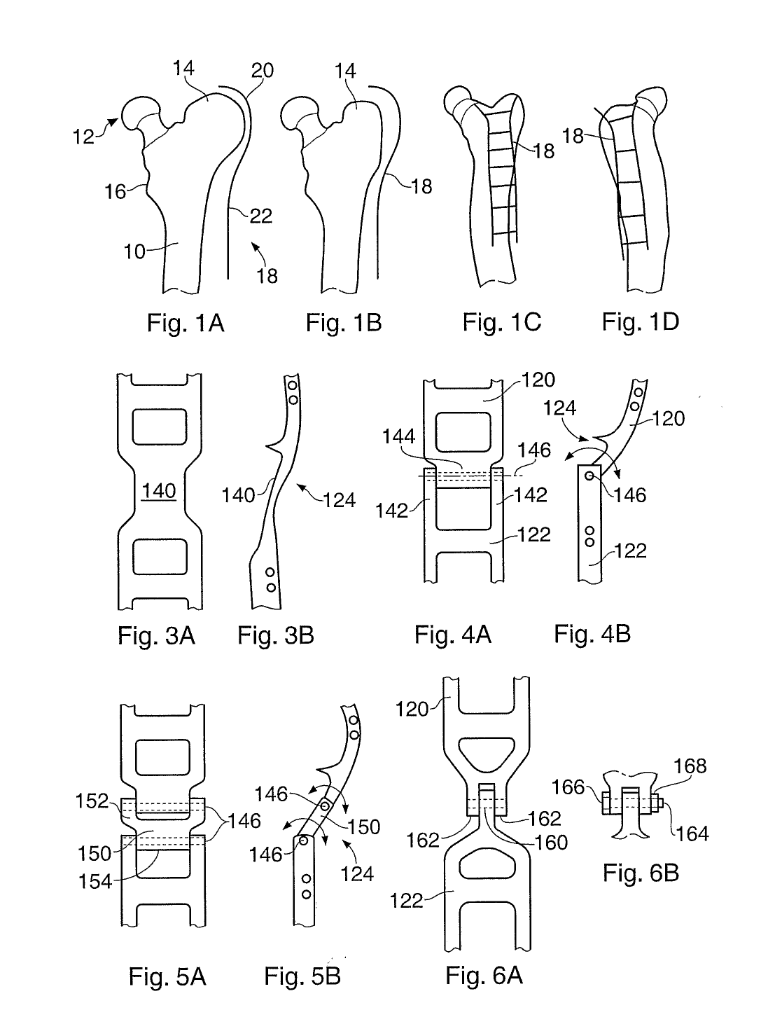

### MODES FOR CARRYING OUT THE INVENTION

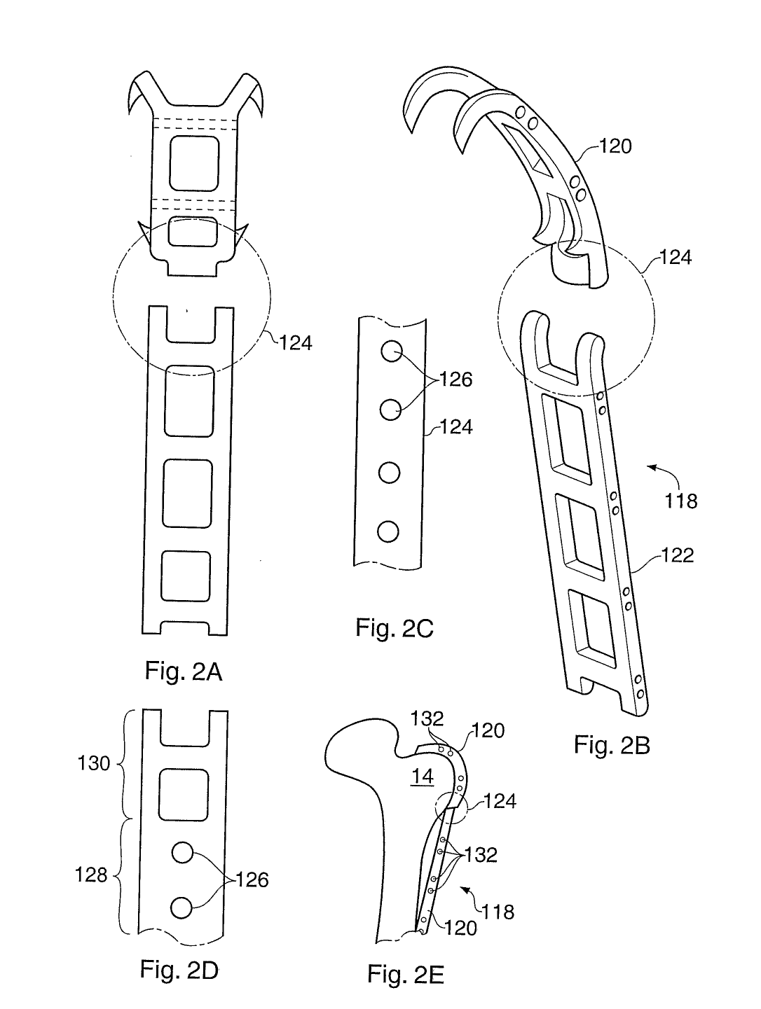

As shown schematically in FIGS. 2A and 2B, the solution proposed in the present application is to produce a grip plate 118 in which the grip portion 120 is connected to the plate portion 122 via a junction 124 which allows the grip portion to be reoriented relative to the plate portion, prior to fixation to the bone, so that the positions of both portions on the bone can be optimised. This is referred to hereafter as a “flexible junction”. This does not necessarily mean that flexing is possible after fixation, which is generally undesirable. This “flexible junction” is shown in FIGS. 2A and 2B merely by a chain-dotted circle.

In FIGS. 2A and 2B, the plate portion 122 has the form of a “ladder plate” as disclosed in patent U.S. Pat. No. 5,665,089 (incorporated herein by reference). The plate portion can take other forms, e.g. any of those disclosed in U.S. Pat. No. 5,665,089. It can be simple plate 124 with screw holes 126 as shown in FIG. 2C, or a combination having a portion 128 with screw holes 126, and a portion 130 formed as a ladder plate, as shown in FIG. 2D.

FIG. 2E shows such a grip plate with a flexible junction 124 mounted on a femur, with the grip portion 120 engaged over the greater trochanter 14, and the plate portion hinged to a suitable angle relative to the grip portion 120 to enable it to lay against the shaft of the femur. Both the grip and plate portions 120, 122 are shown as having through-bores 132. These are intended for receiving cerclage cables. (As described in U.S. Pat. No. 5,665,089 the ladder plate concept enables a plate to be adapted to have various types of fixation means, including provision for cerclage cables, and provision for screw fixing.)

FIGS. 3A and 3B show a simple form of flexible junction 124. In this case, there is a thinned section 140 (see FIG. 3B) which may also be of reduced width (as seen in FIG. 3A). This constitutes a region where the device is capable of flexing.

FIGS. 4A and 4B show a mechanical flexible junction 124. The plate portion 122 has a pair of arms 142 which extend on either side of a tongue portion 144 of the grip portion 120. Transverse apertures are aligned, and a hinge pin 146 passes through them, enabling the grip portion to pivot relative to the plate potion, in the plane of the paper of FIG. 4B. (N.B. whereas it is shown that the grip portion has the tongue and the plate portion has the arms 142, this could be reversed, and this is generally true in the following description.)

FIGS. 5A and 5B show a more sophisticated form of flexible junction 124 based on the same principle as that shown in FIGS. 4A and 4B. In this case, there is an intermediate link member 150 which is hinged by means of hinge pins 146 to both the grip portion and the plate portion. Thus, it has an upper portion with arms 152 like the arms 142 of the plate portion, and a lower portion with a tongue 154 like the tongue of the grip portion. This double linkage gives the device greater freedom to form to a range of shapes of bone. (Of course, a linkage involving a tongue and arms could be reversed so that, e.g., a tongue of an intermediate link member was embraced by arms of a grip portion.)

FIG. 6A shows a variant in which the plate portion 122 has an upperwardly extending narrow tongue 160 which extends between a pair of arms 162 formed on the grip portion 120. Once again, aligned apertures receive a pivot pin or the like. As shown in FIG. 6B, this may be a threaded bolt 164 having a head 166 at one end, and receiving a lock nut 168 on its protruding other end. Thus, a suitable plate portion 122 and grip portion 120 can be selected (e.g. from a range of shapes, sizes and designs) and connected together by means of the nut and bolt. They can be flexed so that they fit the bone to which they are to be connected, and the lock nut can be tightened, to convert the assembly into a substantially rigid unit.

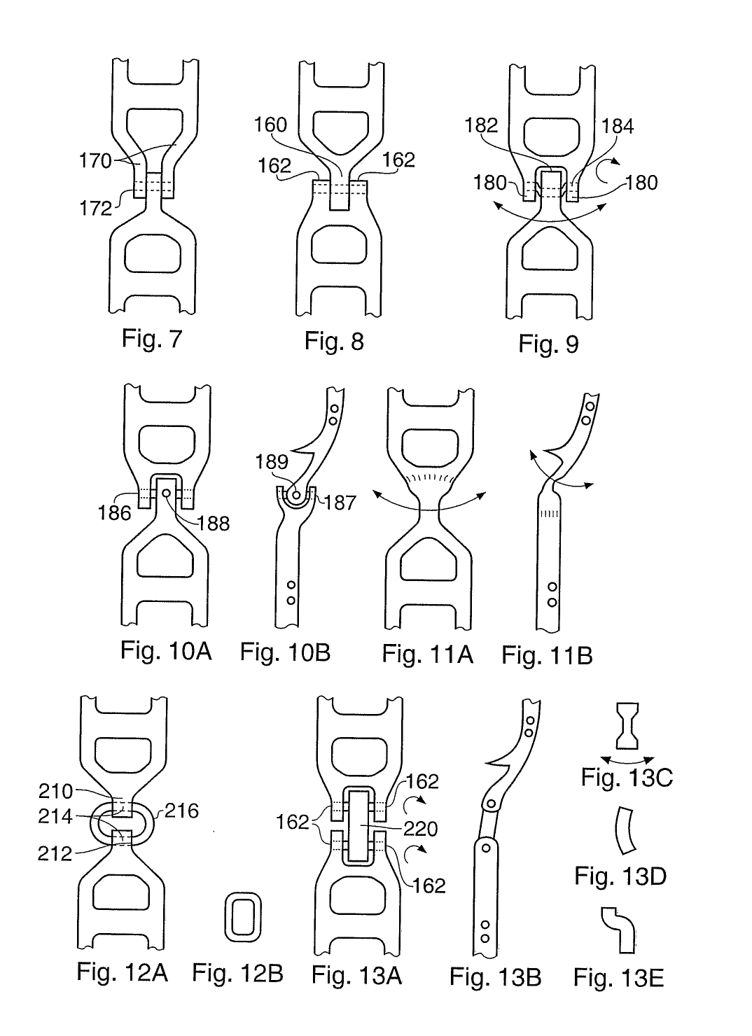

In FIG. 6A, the illustrated part of the grip portion has the form of an inverted rounded “A”, with the projecting arms 162 extending from near the top of the “A”. FIG. 7 shows a variant in which the side bars 170 of the grip portion do not coalesce to form an “A”, but terminate in parallel portions 172 corresponding to the parallel arms 162 shown in FIG. 6A.

FIG. 8 shows an “inverted” version of the design of FIG. 6A, in which the parallel arms 162 are provided by the plate portion, and the tongue 160 is provided by the grip portion.

In the designs shown in FIGS. 6, 7 and 8, a tongue is received quite closely between a pair of arms, and the pivot pin or bolt permits pivoting movement in a single plane. In contrast, FIG. 9 shows a pair of arms whose spacing is significantly greater than the width of the tongue 182. The pivot pin 184 which bridges the arms and passes through an aperture in the tongue 182 is arcuate. The aperture in the tongue 182 is correspondingly arcuate. Thus, the plate portion can both pivot out of the plane of the paper, and, to a limited degree, pivot in the plane of the paper by sliding along the arcuate pivot pin 184.

For still greater freedom of variation of configuration, the grip and plate components may be connected by a universal joint arrangement. For example, FIGS. 10A and 10B show a connection involving orthogonal pairs of yoke arms 186, 187 one pair on each component, and orthogonal pivot pins 188, 189 mounted on respective pairs.

A simpler form of junction permitting universal reconfiguration is shown in FIGS. 11A and 11B, where there is a unitary device, like that shown in FIGS. 3A and 3B, but in which the material is not only thinned as shown in FIGS. 3B and 11B, but it is also narrowed to such an extent (as seen in FIG. 11A) that bending is also possible in the plane of the paper.

FIG. 12A shows another type of linkage giving considerable freedom. In this case, the grip and plate portions have opposed projecting tongues 210, 212, each having a transverse bore 214. A linking ring 216 passes through both of the bores 214, thus permitting relative pivoting out of the plane of paper, as well as swivelling around the ring. Other shapes of connecting element are possible, e.g. oval or rounded-rectangular as shown in FIG. 12B.

FIG. 13A shows a device which includes a grip portion as in FIG. 6A and a plate portion as in FIG. 8, each having a spaced pair of arms 162. In the arrangement shown in FIG. 13A, they are linked by a link pin 220 having near each end an aperture for a pivot pin or locking bolt. FIGS. 13C, 13D and 13E show some variants of the link pin 220. FIG. 13C shows a waisted link pin, capable of being bent in its thinned central region.

FIG. 13D shows an arcuate link pin which can be mounted in either orientation, so that the assembled plate and grip portions have a rightward or leftward twist. FIG. 13E shows a pin with offset parallel end regions, allowing offsetting of the grip and plate portions.

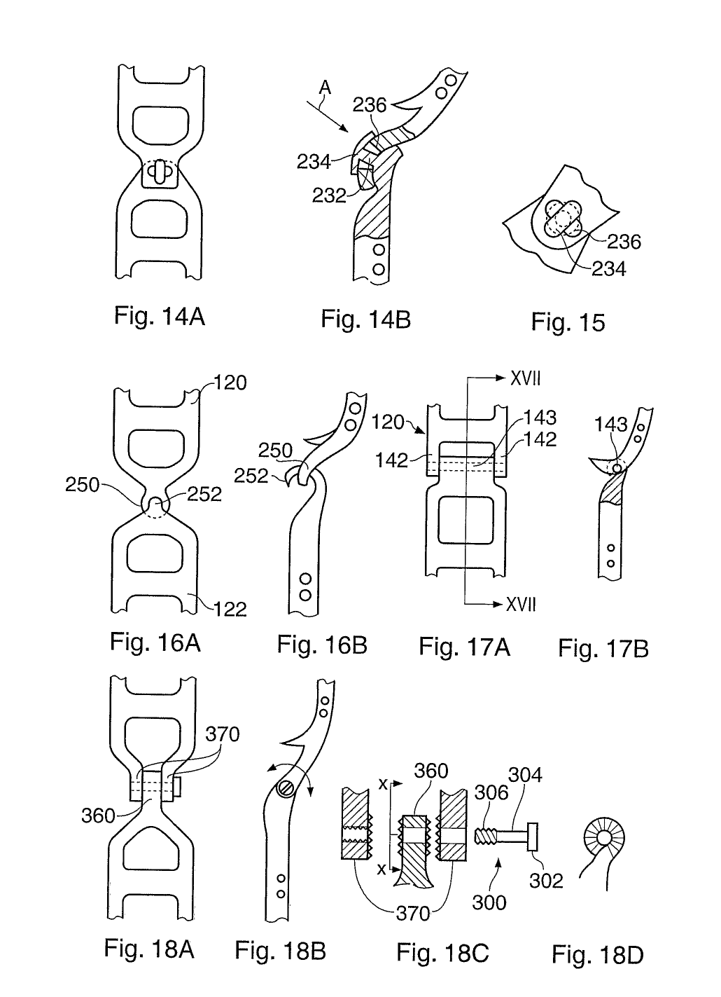

FIGS. 14A, 14B and 15 show grip and plate portions which can be mutually engaged and disengaged. One component has a projection 232 bearing an elongated head 234. The other component has a slot 236 whose mouth is narrowed so that it is elongated, such that the head can be passed through it only when the components are in the correct relative orientation. In the normal use orientation as shown, the head cannot be removed from the slot and thus the components are pivotably connected together.

FIGS. 16A and 16B show an arrangement in which one component has a loop or eye 250, which is pivotably engagable by a hook 252 of the other components.

FIGS. 17A and 17B show a more robust hook design in which one component (here the grip portion 120) provides a spaced pair of arms 142 through which a hinge pin extends, as shown for the plate portion 122 in FIG. 4. Whereas in FIG. 4, the other component has an aperture through which the hinge pin is threaded, in FIG. 17, the other component has a hook 143 which is engageable with and disengageable from the pin.

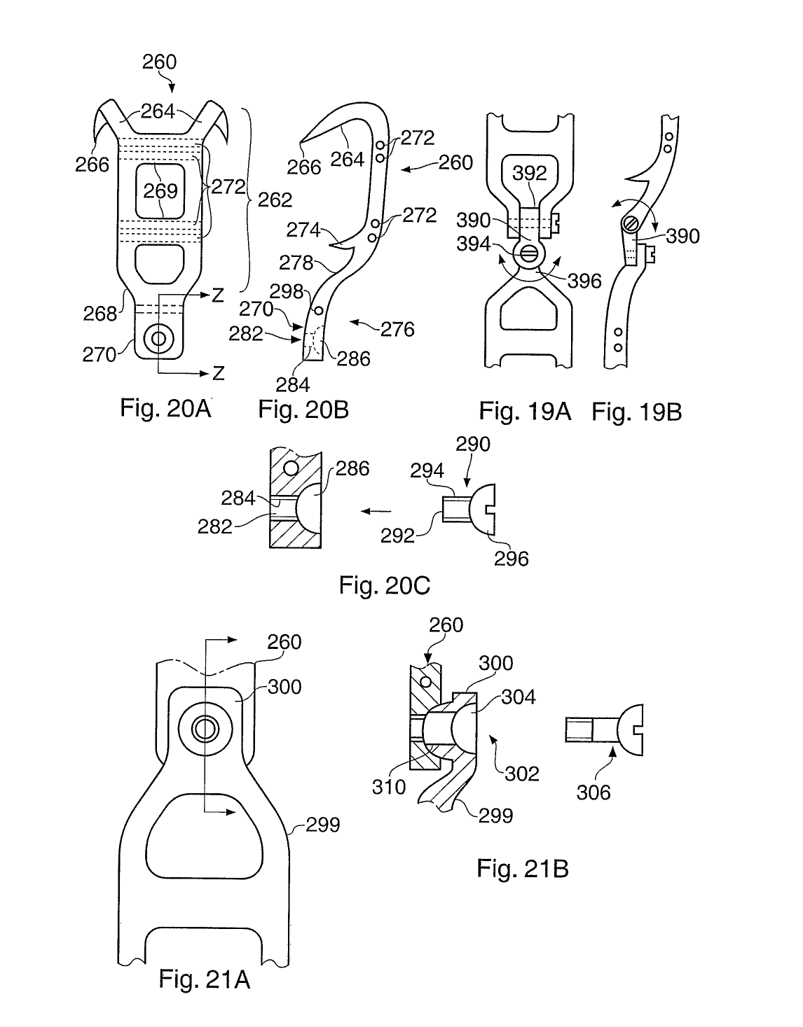

FIGS. 18A, 18B, 18C and 18D show a design similar to that of FIG. 7, but with a locking screw in place of the simple hinge pin. The screw 300 has a head 302, a shank 304 and a threaded end portion 306. Side bars 370 of the grip portion embrace a tongue 360 of the plate portion, with apertures in register to receive the locking screw 300. The aperture in one side bar is threaded to engage the threaded end portion 306 of the screw. The opposing faces of the side bars 370 and tongue 360 have radial projections 380 (see FIG. 19D) so that, when they are urged together by tightening the screw 300, relative rotation is prevented.

FIGS. 19A and 19B show a related design with two lockable pivotting connections. It includes an intermediate link member 390 having an upper portion 392 formed like the tongue 360 and coupled to side bars 370 of the grip portion, and a lower portion 394 formed like the side bars 370 and coupled to a tongue 396 on a plate portion. However the aperture in this tongue 396 is orthogonal to the main plane of the plate portion, so that the two connections enable pivotting in orthogonal planes.

FIGS. 20A, 20B and 20C show a bone grip 260 which can be coupled to a plate portion, e.g. as described above, or used separately. Its main or bone engagement portion 262 may be substantially the same as a known type of bone grip. Thus as illustrated, there is a pair of side limbs 264 which curve rearwardly at one end, terminating in sharp hook ends 266. At the other end 268, the limbs merge. One or more cross pieces 269 join the limbs and are penetrated by transverse bores 272 for cerclage cables. As shown in FIG. 20B, there may be lower hook portions 274 adjacent the lower end 268. In this new design, the lower end 268 is prolonged by a projecting connection portion 276. As seen in FIG. 20B, this may be arcuate so that it can end with a portion able to overlie the proximal shaft of a femur. It may be joined to the bone engagement portion 262 by a “hingeing region”, shown in FIG. 20B as a portion 278 of reduced thickness. The connection portion 270 has an aperture 282 with a threaded portion 284 and an outer enlarged (countersunk) portion 286. As shown in FIG. 20C, a locking bone screw 290 having a unicortical or bicortical screw tip 292 (for anchoring to bone), a threaded shaft 294 complementary to the threaded portion 284, and a head 296 receivable in the enlarged portion 286 can be engaged in the aperture to assist in fixing the bone grip 260 to bone. (Alternatively a non-locking screw could be used, whereof the portion which extends through the threaded aperture portion 284 is of lesser diameter and does not engage it threadedly.) The connection portion 270 may also have one or more transverse bores 298 for distal cerclage cables.

FIGS. 21A and 21B show how the bone grip 260 can be coupled to a plate portion 299. This is provided with an upper tongue 300 with an aperture 302 with an enlarged (countersunk) portion 304. Thus a locking screw 306 can be passed partially through the aperture 302 and engage the threaded portion 284 of the aperture in the connection portion 270 of the bone grip 260.

As shown in FIGS. 21A and 21B, the tongue 300 preferably has a curved rearward bulge 310 concentric with the aperture 302, which fits into the enlarged portion 286. This permits multiaxis alignment, prior to locking with the locking screw 306 or use with a unicortical or bicortical bone screw.

The various components can be made from any suitable surgical materials. Most commonly, they will be of metal, e.g. cobalt chrome, suitable stainless steels, or titanium alloys. Alternatively they may comprise polymeric (plastic) materials. In the case of embodiments depending on bending of a “live hinge” region, clearly the hinging region must be made of a material that is capable of bending, possibly after being annealed. In some cases, polymeric (plastic) materials may be suitable.

Whereas the invention has been exemplified by components for use in hip replacement operations, it is also applicable to surgical components used in other sites and contexts. Thus the grip portion could instead be a device as generally known for (e.g.) fixation of multipart fractures, or alignment osteotomies in the extremities of long bones.

1) A bone fixation component comprising an elongate first portion for engaging over an elongate bone, and an end engagement second portion for engaging at a bone end region, characterised in that the first portion is hingedly connected or connectable to said second portion.

2) A component according to claim 1 wherein the hinged connection permits hingeing in the medio-lateral and antero-posterior planes.

3) A component according to claim 1 wherein the first and second portions are connected or connectable via a connecting region which permits bending to provide the hinged connection.

4) A component according to claim 1 wherein the first and second portions are connected or connectable via a connecting region providing one or more pivoting connections.

5) A component according to claim 4 wherein said pivoting connection (s) are lockable to restrain pivoting thereof.

6) A component according to claim 4 wherein each of said components has a projecting portion, and, in use, the projecting portions overlap and are pivotably coupled.

7) A component according to claim 6 wherein one of said component portions has a pair of spaced projecting portions extending, in use, on either side of the projecting portion of the other component portion and being pivotably coupled to it.

8) A component according to claim 4 including an intermediate element connected or connectable to said first and second portions via respective pivoting connections.

9. A component according to claim 1 wherein the first portion is a ladder plate having a pair of elongate side rails bridged by a plurality of bridge portions.

10) A component according to claim 9 wherein the ladder plate has formations for engaging or guiding cerclage cables.

11) A component according to claim 1 wherein the second portion is a bone grip.

12) A kit of parts for producing a component according to claim 1, comprising a plurality of different first portions and a plurality of different second portions.

13) A bone grip having an arcuate hook portion for engaging over a bone end region and a projecting tongue portion which in use projects along the bone and is adapted to receive selectively either a bone fixation screw for securing it to bone, or a coupling element for coupling it to an elongate first portion to form a component according to claim 1.

14. A method of bone fixation comprising: (a) providing a component according to claim 1 with the first and second portions hingedly connected; and (b) effecting hingeing to adapt the component to an intended bone site. 1) A bone fixation component comprising an elongate first portion for engaging over an elongate bone, and an end engagement second portion for engaging at a bone end region, characterised in that the first portion is hingedly connected or connectable to said second portion.
